(19) United States
(12) Patent Application Publication
(10) Pub. No.: US 2026/0201482 A1
(43) Pub. Date: Jul. 16, 2026

(54) METHOD OF PREDICTING MICROBIAL CONTAMINATION

(71) Applicant: Cargill, Incorporated, Wayzata, MN (US)

(72) Inventors: Ted Brown, Valley Center, KS (US); Melody Thompson, Wichita, KS (US)

(73) Assignee: Cargill, Incorporated, Wayzata, MN (US)

(21) Appl. No.: 19/135,871

(22) PCT Filed: Dec. 11, 2023
(86) PCT No.: PCT/US2023/083325

§ 371 Date: Jun. 5, 2025

Related U.S. Application Data
(60) Provisional application No. 63/476,419, filed on Dec. 21, 2022.

(51) Int. Cl. C12Q 1/689; A23B 2/05; A23B 2/10; A23B 2/40; A23B 2/50; A23B 4/005; A23B 4/01; A23B 4/015; A23B 4/14

(52) U.S. Cl. CPC C12Q 1/689; A23B 2/05; A23B 2/10; A23B 2/40; A23B 2/50; A23B 4/0053; A23B 4/01; A23B 4/015; A23B 4/14

(57) ABSTRACT

A method of predicting microbial contamination includes gathering a sample from one or more carcasses. The method includes performing a test for the microbial contamination on the sample to generate test results including detection of one of more microbial genetic markers of the of the microbial contamination. The method includes predicting from the test results whether the one or more carcasses include the microbial contamination. The method includes releasing the one or more carcasses for further processing.

### CROSS-REFERENCE TO RELATED APPLICATIONS

This application claims the benefit of U.S. Provisional Application No. 63/476,419, filed Dec. 21, 2022, which is incorporated by reference herein in its entirety.

### BACKGROUND

Escherichia coli O157:H7 has been a pathogen of concern to the meat processing industry for decades. Numerous outbreaks of hemorrhagic colitis caused by E. coli O157:H7 were associated with consumption of ground beef in the United States. These outbreaks events led the Food Safety and Inspection Service (FSIS) to declare the E. coli O157:H7 and other STEC organisms an adulterant in beef and to require that meat processors establish hazard analysis and critical control point (HACCP) plans for their plants. The beef industry loses millions due to down-graded beef product per year due to E. coli 0157 testing. Commercial processing plants utilize, numerous intervention strategies focusing on prevention of carcass contamination and decontamination of carcasses. Beef processing plants presently employ several interventions (e.g., trimming, steam pasteurization, water washes, and organic acid washes) in combination to achieve large reductions in carcass contamination in accordance with their individual HACCP plans. Beef processing plants verify their intervention processes are in control by testing for pathogens. However, due to very low prevalence rate and the low numbers of bacterial cells present, this approach is limited in the ability to predict or reduce contamination with, or to verify intervention processes have reduced, pathogens such as E. coli O157:H7.

Indicator organism testing such as via aerobic plate count (APC) or Enterobacteriaceae testing can be used to provide statistically significant data for analysis; however, frequently, a weak relationship between the indicator organism(s) and the pathogen limits the ability of indicator organism testing to predict or reduce contamination with, or to verify intervention processes have reduced, pathogens such as E. coli O157:H7.

### SUMMARY OF THE INVENTION

Various aspects of the present invention provide a method of predicting microbial contamination. The method includes gathering a sample from one or more carcasses. The method includes performing a test for the microbial contamination on the sample to generate test results. The test results include detection one or more microbial genetic markers of the microbial contamination. The method includes predicting from the test results whether the one or more carcasses include the microbial contamination. The method also includes releasing the one or more carcasses for further processing.

Various aspects of the present invention provide a method of predicting microbial contamination. The method includes gathering a sample from one or more carcasses, including swabbing an exterior of the one or more carcasses. The method includes performing a test for the microbial contamination on the sample to generate test results including detection of one or more microbial genetic markers of the microbial contamination, wherein the one or more microbial genetic markers of the microbial contamination includes one or more genetic markers from one or more gram negative organisms in the Enterobacteriaceae family other than the predicted microbial contamination. The method includes predicting from the test results whether the one or more carcasses include the microbial contamination. The method also includes releasing the one or more carcasses for further processing.

Various aspects of the present invention provide a method of predicting microbial contamination from Escherichia coli O157:H7 (E. coli O157:H7). The method includes gathering a sample from one or more carcasses, including swabbing an exterior of the one or more carcasses. The method includes performing a test for the microbial contamination on the sample to generate test results including detection of one or more microbial genetic markers of the microbial contamination, wherein the one or more microbial genetic markers of the microbial contamination include one or more genetic markers from one or more gram negative organisms in the Enterobacteriaceae family other than E. coli O157:H7. The method includes predicting from the test results whether the one or more carcasses include the microbial contamination. The method also includes releasing the one or more carcasses for further processing.

In various aspects, the method of the present invention can predict microbial contamination in carcasses with greater accuracy than other methods, such as compared to methods that culture pathogens or methods that use indicator organism testing. In various aspects, the method of the present invention can predict microbial contamination in carcasses with greater sensitivity, greater specificity, greater speed, lower cost, less wasted carcasses, or a combination thereof, as compared to other methods, such as compared to methods that culture pathogens or methods that use indicator organism testing. In various aspects, the method of the present invention can reduce the amount of finished product testing, the amount of finished product lost due to carcass contamination detection, or a combination thereof, as compared to other methods, such as compared to methods that culture pathogens or methods that use indicator organism testing.

### BRIEF DESCRIPTION OF THE FIGURES

The drawings illustrate generally, by way of example, but not by way of limitation, various aspects of the present invention.

FIG. 1 is a plot illustrating number of S-factor positives versus daily average APC, in accordance with various aspects of the present invention.

FIGS. 2A-D illustrate testing of S-factor at slaughter vs. testing of O157 at processing, with FIG. 2A illustrating a relationship of prevalence (p, proportion of contaminated samples), sensitivity (Se, probability of test positive with contaminated sample), and specificity (Sp, probability of a test negative with a non-contaminated sample) to sampling and testing methodology, FIG. 2B illustrates a testing grid showing days with one or more positive S-samples at pre-evisceration 2-3 days prior to processing for O157 positive or negative samples, FIG. 2C illustrates a sensitivity of 100% (95% Crl: 46.4-100), and FIG. 2D illustrates a specificity of 13% (95% Crl: 3.8-30.9), in accordance with various aspects of the present invention.

FIGS. 3A-C illustrate testing of S-factor at slaughter vs. testing of STX2 at processing, with FIG. 3A illustrating a testing grid showing days with one or more positive S-samples at pre-evisceration 2-3 days prior to process for STX 2 positive or negative samples, FIG. 3B illustrating a sensitivity of 86.6% (95% Crl: 63.6-97.1), and FIG. 3C illustrating a specificity of 9.1% (95% Crl: 1.0-35.3), in accordance with various aspects of the present invention.

FIGS. 4A-C illustrate testing of S-factor vs. testing of O157 at processing, with FIG. 4A illustrating a testing grid showing days with one or more positive S-samples at processing for O157 positive or negative samples, FIG. 4B illustrating a sensitivity of 100% (95% Crl: 46.4-100), and FIG. 4C illustrating a specificity of 30.4% (95% Crl: 14.8-50.7), in accordance with various aspects of the present invention.

FIGS. 5A-C illustrate testing of S-factor vs. testing of STX2, with FIG. 5A illustrating a testing grid showing days with one or more positive S-samples at processing for STX 2 positive or negative samples, FIG. 5B illustrating a sensitivity of 80% (95% Crl: 55.6-94.0), and FIG. 5C illustrating a specificity of 36.3% (95% Crl: 13.7-65.2), in accordance with various aspects of the present invention.

FIGS. 6A-C illustrate testing of S-factor vs. testing of O157 at processing using wet pool samples, with FIG. 6A illustrating a testing grid showing days with one or more positive wet pool S-samples at processing for wet pool O157 positive or negative samples, FIG. 6B illustrating a sensitivity of 16.6% (95% Crl: 1.8-55.8), and FIG. 6C illustrating a specificity of 95.3% (95% Crl: 97.8-98.7), in accordance with various aspects of the present invention.

FIGS. 7A-C illustrate testing of S-factor vs. testing of STX2 at processing using wet pool samples, with FIG. 7A illustrating a testing grid showing days with one or more positive wet pool S-samples at processing for wet pool STX2 positive or negative samples, FIG. 7B illustrating a sensitivity of 2.8% (95% Crl: 0.3-12.2), and FIG. 7C illustrating a specificity of 98.3% (95% Crl: 97.7-98.7), in accordance with various aspects of the present invention.

FIGS. 8A-C illustrate testing of S-factor vs. testing of O157/STX2 at processing, as follow-up testing on individuals from positive wet pool, with FIG. 8A illustrating a testing grid showing days with high or medium/negative magnitude individual S-factor for O157/STX2 positive or negative samples, FIG. 8B illustrating a sensitivity of 100% (95% Crl: 14.6-100), and FIG. 8C illustrating a specificity of 52.7% (95% Crl: 23.4-48.4), in accordance with various aspects of the present invention.

FIGS. 9A-C illustrate testing of STX2 versus O157, with FIG. 9A illustrating a testing grid showing days with at least one positive STX2 at processing for O157 positive or negative samples, FIG. 9B illustrating a sensitivity of 100% (95% Crl: 46.4-100), and FIG. 9C illustrating a specificity of 47.8% (95% Crl: 28.7-67.4), in accordance with various aspects of the present invention.

FIGS. 10A-C illustrate testing of STX2 versus O157 using wet pool samples, with FIG. 10A illustrating a testing grid showing days with one or more positive wet pool S-samples at processing for O157 positive and negative samples, FIG. 10B illustrating a sensitivity of 100% (95% Crl: 66.9-100), and FIG. 10C illustrating a specificity of 98.8% (95% Crl: 98.2-99.2), in accordance with various aspects of the present invention.

FIG. 11 illustrates days with one or more positive test using various testing protocols versus sensitivity, in accordance with various aspects of the present invention.

FIG. 12 illustrates sensitivity versus 1-specificity (e.g., false positive rate), in accordance with various aspects of the present invention.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Method of Predicting Microbial Contamination

Various aspects of the present invention provide a method of predicting microbial contamination. The method can include gathering a sample from one or more carcasses. The method can include performing a test including detection of one or more microbial genetic markers of the microbial contamination. The method can include predicting from the test results whether the one or more carcasses include the microbial contamination. The method can also include releasing the one or more carcasses for further processing.

The carcass can be a carcass of any suitable animal, such as a beef carcass, a poultry carcass, a pork carcass, a fish carcass, or a lamb carcass. The carcass can be a beef carcass. As contrasted with trim pieces, the carcass can be a whole carcass, such as a whole carcass at various stages of processing.

The one or more carcasses from which the sample is gathered can include a hide. The hide can be an open hide. The one or more carcasses from which the sample is gathered can be free of hide.

The one or more carcasses from which the sample is gathered can be pre-eviscerated. The one or more carcasses from which the sample is gathered can be eviscerated.

The one or more carcasses from which the sample is gathered can be trimmed. The one or more carcasses can be free of trim pieces.

The method can include gathering a sample from one or more carcasses. The sample can be gathered in any suitable way. Gathering the sample can include gathering the sample from an exterior of the one or more carcasses. Gathering the sample can include gathering the sample from a surface area of 50 cm² to 10,000 cm² of a surface area of the exterior of the one or more carcasses, such as less than or equal to 10,000 cm²and greater than or equal to 50 cm², 100, 250, 500, 1000, 1500, 2000, 2500, 5000, 7500, or 9000 cm². Gathering the sample can include gathering the sample from 1% to 95% of a total surface area of the exterior of the one or more carcasses, such as less than or equal to 95% and greater than or equal to 1%, 2, 4, 6, 10, 15, 20, 30, 40, 50, 60, 70, 80, or 90%. Gathering the sample can include gathering the sample from a hide on the one or more carcasses, a de-hided portion of the one or more carcasses, an eviscerated portion of the one or more carcasses, a trimmed portion of the one or more carcasses, or a combination thereof.

Gathering the sample can include swabbing an exterior of the one or more carcasses. The swabbing can be performed with any suitably absorbent material. For example, gathering the sample can include swabbing an exterior of the one or more carcasses with one or more sponges. Gathering the sample can be performed solely on the exterior of the one or more carcasses. Gathering the sample can be free of gathering the sample from an interior of the one or more carcasses. The method can be free of gathering the sample, or any sample, from an interior of any of the one or more carcasses. The gathering the sample can consist of gathering the sample from an interior of the one or more carcasses.

The sample can include a sample from one carcass and not more than one carcass. The sample can include a sample from more than one carcass; for example, the sample can include a sample from more than one carcass gathered in parallel, a sample from more than one carcass gathered in series, or a combination thereof.

In various aspects, a production lot includes the one or more carcasses from which the sample is gathered. The one or more carcasses from which the sample is gathered can be first one or more carcasses, and the method can further include performing the method on a second one or more carcasses in the production lot. The method can include performing the method on all the carcasses in the production lot (e.g., gathering samples from all carcasses in the production lot). The method can include performing the method on less than all the carcasses in the (e.g., gathering samples from less than all carcasses in the production lot).

The method can include maintaining and testing the sample from the first one or more carcasses and a sample from the second one or more carcasses. The method can be free of combining the sample from the first one or more carcasses and the sample from the second one or more carcasses. For example, the sample from the first one or more carcasses can be collected with one swab, and the sample from the second one or more carcasses can be collected with another swab.

Gathering the sample from the second one or more carcasses in the production lot can include combining the sample from the second one or more carcasses in the production lot with the sample from the first one or more carcasses in the production lot. Gathering the sample from the second one or more carcasses in the production lot can include swabbing an exterior of the second one or more carcasses in the production lot used to swab an exterior of the first one or more carcasses in the production lot.

The method of predicting microbial contamination can be used to predict any suitable type of microbial contamination. For example, the microbial contamination can include bacterial, fungal, viral, and/or parasitic pathogens; indicator organisms (e.g., heterotrophes, generic E. coli, and/or total and fecal coliforms and enterococcus); spoilage organisms (e.g., Pseudomonas); indicator molecules (e.g., glial fibillary acid protein (GFAP)); transmissible spongiform encephalopathy (TSE) agents (prions) (e.g., bovine spongiform encephalopathy (BSE) agents, scrapie, and/or chronic wasting disease); and/or combinations thereof. The microbial contamination can include Escherichia coli O157:H7 (E. coli O157:H7), enterohemorrhagic Escherichia coli (EHEC), enterotoxigenic Escherichia coli (ETEC), enteroinvasive Escherichia coli (EIEC), enterpathogenic Escherichia coli (EPEC), Salmonella, Listeria, Yersinis, Campylobacter, Staphylococcus spp., and/or Clostridial species. The microbial contamination can include Escherichia coli O157:H7 (E. coli O157:H7).

The method can include performing a test including detection of one or more microbial genetic markers of the microbial contamination. For example, the test can include one or more PCR assays. The one or more microbial genetic markers of the microbial contamination that are detected can be genetic markers from the microbial contamination (e.g., genetic markers from the one or more microbes that comprise the microbial contamination, such as one or more genetic markers from E. coli O157:H7), from one or more microorganisms whose presence is indicative of the microbial contamination, or a combination thereof. Detecting the one or more microbial genetic markers can include determining whether the one or more microbial genetic markers are present in the sample. The one or more microorganisms whose presence is indicative of the microbial contamination can be any suitable one or more microorganisms that together are indicative of the microbial contamination, such as one or more gram negative organisms in the Enterobacteriaceae family. The one or more gram negative organisms can be other than the microbial contamination being predicted (e.g., if the microbial contamination includes E. coli O157:H7, then the one or more gram negative microorganisms to which the one or more genetic markers belong can be other than E. coli O157:H7).

The test for the microbial contamination can include a test that detects more than one microbial genetic marker of the microbial contamination. The test for the microbial contamination can include a test that detects one and not more than one microbial genetic marker of the microbial contamination. For example, the test for the microbial contamination can include a test that detects one or more microbial genetic markers of Escherichia coli O157:H7 (E. coli O157:H7) (e.g., wherein the genetic markers are from E. coli, from one or more microorganisms indicative of the microbial contamination such as one or more gram negative microorganisms in the Enterobacteriaceae, or a combination thereof). The test for the microbial contamination can include a test that detects one and not more than one microbial genetic marker of Escherichia coli O157:H7 (E. coli O157:H7).

The one or more microbial genetic markers of the microbial contamination can include one or more genetic markers from a single microorganism that is indicative of the microbial contamination. The one or more microbial genetic markers of the microbial contamination can include one or more genetic markers from more than one microorganism that is indicative of the microbial contamination. The one or more microbial genetic markers of the microbial contamination can include a genetic marker from one or more microorganisms that are indicative of microbial contamination with Escherichia coli O157:H7 (E. coli O157:H7). The one or more microorganisms that are indicative of microbial contamination with Escherichia coli O157:H7 can include one or more gram negative organisms in the Enterobacteriaceae family other than Escherichia coli O157:H7.

The test for the microbial contamination can include an S-target test, a PCR assay for O157, a PCT assay for STX2, or a combination thereof.

The method can include predicting from the test results whether the one or more carcasses include the microbial contamination. Predicting whether the one or more carcasses include the microbial contamination can include predicting that the one or more carcasses include the microbial contamination if the results of the test indicate presence of the one or more microbial genetic markers of the microbial contamination in the sample, and predicting that the one or more carcasses do not include the microbial contamination if the results of the test indicate no detectable presence of the one or more microbial genetic markers of the microbial contamination. Predicting whether the one or more carcasses include the microbial contamination can include using a correlation between the test for the microbial contamination and a positive test for the microbial contamination from a different test for the microbial contamination to predict whether the test results indicate that the one or more carcasses include the microbial contamination. The method can further include generating the correlation between the test for the microbial contamination and the positive test for the microbial contamination from the different test from the microbial contamination.

The correlation can include a percent certainty based on a degree of positivity of the detection of the one or more microbial genetic markers, wherein predicting whether the one or more carcasses includes the microbial contamination includes predicting that the test results indicate that the one or more carcasses include the microbial contamination if the percent certainty of the correlation indicated by the positivity of the detection of the one or more microbial genetic markers equals or exceeds a pre-determined threshold percent certainty, and predicting that the test results indicate that the one or more carcasses do not include the microbial contamination if the percent certainty of the correlation indicated by the positivity of the detection of the one or more microbial genetic markers is less than the pre-determined threshold percent certainty.

The method can also include releasing the one or more carcasses for further processing. Releasing the one or more carcasses for further processing can include performing bacterial reduction processing on the one or more carcasses if the one or more carcasses are predicted to include the microbial contamination, and optionally can include performing bacterial reduction processing on the one or more carcasses if the one or more carcasses are predicted to not include the microbial contamination. Performing bacterial reduction processing can include cooking, irradiation, high pressure processing, thermal carcass surface treatment, steam carcass surface treatment, hot water carcass surface treatment, carcass surface treatment with one or more antimicrobial compounds, or a combination thereof. Releasing the one or more carcasses for further processing can include performing further testing for the microbial contamination on the one or more carcasses or processing products thereof and optionally can include releasing the one or more carcasses into a stream of commerce for consumption if the further testing indicates absence of the microbial contamination. Releasing the one or more carcasses for further processing can include releasing the one or more carcasses into a stream of commerce for consumption. Releasing the one or more carcasses for further processing can include fabricating the one or more carcasses into raw products. The method can be free of holding carcasses based on the test that includes detection of the one or more microbial genetic markers of the microbial contamination.

### EXAMPLES

Various aspects of the present invention can be better understood by reference to the following Examples which are offered by way of illustration. The present invention is not limited to the Examples given herein.

### Example 1. Validation Trial. Protocol Study of S-Target Compared to E. Coli O157:H7 Through a Beef Processing Facility

In this study, counts of indicator organisms (aerobic bacteria and Enterobacteriaceae), S-target and E. coli O157 and the prevalence of E. coli O157 are assessed at various steps in carcass processing to identify relationships that may be applied to process control monitoring. The objectives of this study are twofold: (i) to determine the relationship between S-target and E. coli O157 and (ii) to develop a program that can utilize the relationship to monitor the carcass sanitary dress procedures.

### Materials and Methods

Samples were collected over a 12-week period at two commercial fed-beef processing plants, one in Dodge City, KS and the other in Ft. Morgan, CO. The carcass samples were collected on program cattle to limit the number of sorts in the cooler to reduce the mixing of cattle lots. Targeting program cattle ensures the same cattle are measured at the carcasses and the trim samples. Ten samples per week were collected from each of three sample sites during the three months, for a total of 260 samples per site. Samples were collected as described in the carcass mapping procedure at three locations on the processing line: point 1: hide, sampled after hide opening but before hide removal; point 2: pre-evisceration, immediately following de-hiding; point 3: post-evisceration, after evisceration, carcass trimming, and final inspection before the final carcass washes. Individual animals and carcasses were tagged and tracked throughout the process. The same carcasses were sampled at each of the sampling points. During this same three-month period, all program cattle lots sampled for carcass S-target were also analyzed for APC and EB, and all trim lot samples analyzed for E. coli O157:H7 and STX2 were also analyzed for S-target, APC, and EB.

### Sample Collection

All samples were obtained using SpeciSponges (Nasco, Fort Atkinson, Wis.) moistened with 10 mL of buffered peptone water (Difco Laboratories, Sparks, Md.). Excess liquid was squeezed from the sponge into the sponge bag prior to sampling and then used to swab the hide or carcass. The hide sample was collected from a 100-cm² area over the plate region, using a 100-cm² template and flipping the sponge over midway through taking the sample. One sample includes approximately five vertical and five horizontal passes (up and down or side to side will be considered one pass). For each of the two carcass processing points, two separate 4,000-cm² areas were sampled and analyzed separately. Sampling at the pre-evisceration and post-evisceration was facilitated by using two sponges, each moistened with 10 mL of buffered peptone water. One sponge will be used for the inside and outside round area (carcass high) and one for the navel-plate-brisket-foreshank area (carcass low). All sample bags were held refrigerated (2-10° C.) prior to analysis. Sampled carcasses were not held.

### Sample Analysis

The testing laboratory added an additional 10 mL of Buffered Peptone Water to each sponge sample prior to plating. Each sponge was analyzed for total aerobic plate counts (APC), Enterobacteriaceae (EB), E. coli O157:H7, and S-target. APC and EB analysis were performed using 3M petrifilm, E. coli O157:H7 analysis was performed using the Hygiena BAX E. coli exact assay with quant (KIT 2039), and S-target analysis was performed using the Hygiena BAX S-target assay. Fresh combo trim samples were analyzed as stated in the facility procedures but were analyzed for S-target using the Hygiena BAX S-target assay. The genetic target for shinga toxin type 2(STX2) was analyzed using a BioControl Assurance MPX7 PCR assay.

### Statistical Analysis

To compare the prevalence of E. coli O157 between sampling sites, continuity-adjusted chi-square P-values were calculated. APC data was log-transformed before analysis of variance (ANOVA). For each sampling site, a one-way ANOVA was conducted to determine whether counts differed between plants.

### Results

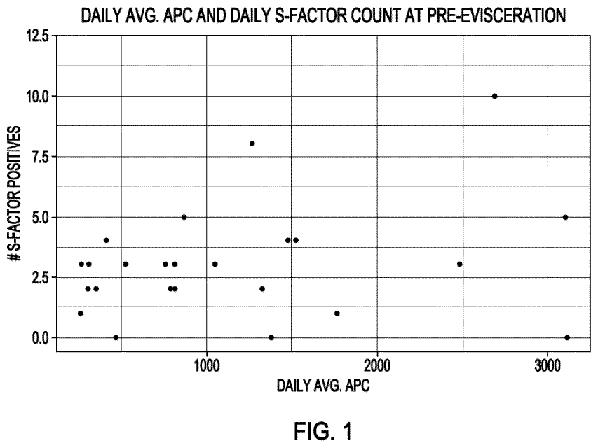

FIG. 1 is a plot illustrating number of S-factor positives versus daily average APC.

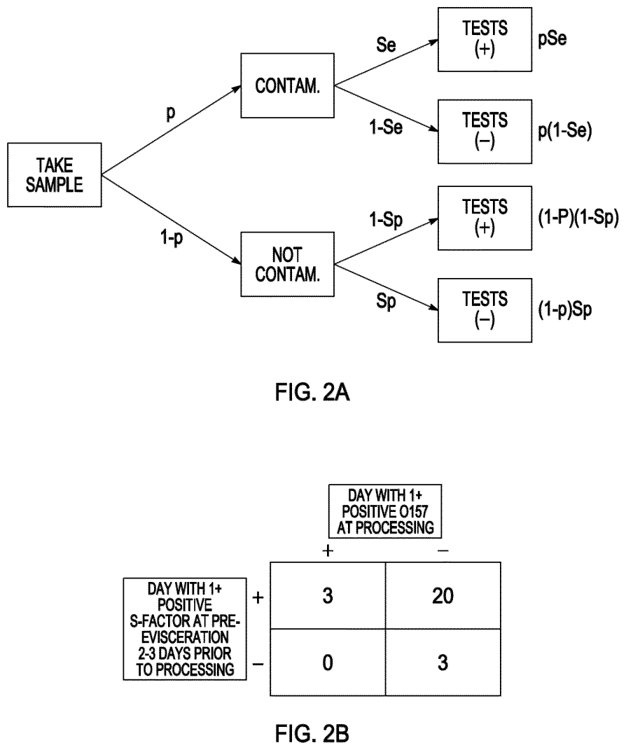

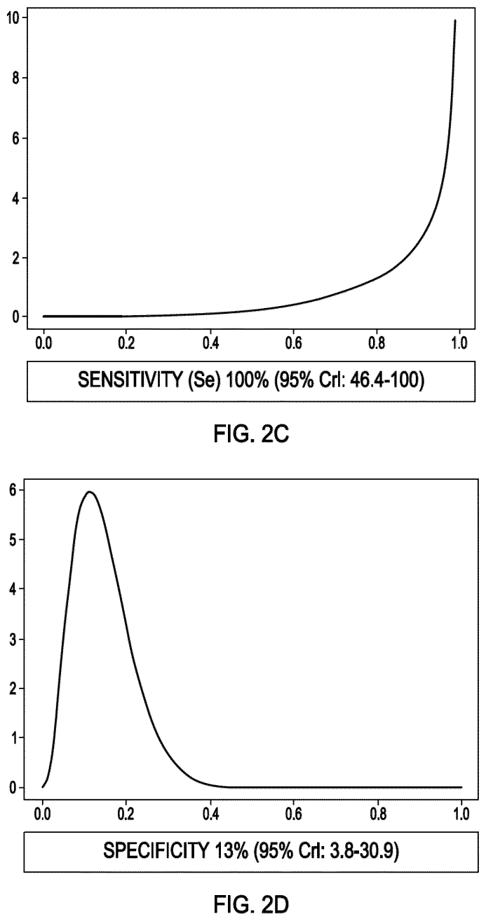

FIGS. 2A-D illustrate testing of S-factor at slaughter vs. testing of O157 at processing, with FIG. 2A illustrating a relationship of prevalence (p, proportion of contaminated samples), sensitivity (Se, probability of test positive with contaminated sample), and specificity (Sp, probability of a test negative with a non-contaminated sample) to sampling and testing methodology, FIG. 2B illustrates a testing grid showing days with one or more positive S-samples at pre-evisceration 2-3 days prior to processing for O157 positive or negative samples, FIG. 2C illustrates a sensitivity of 100% (95% Crl: 46.4-100), and FIG. 2D illustrates a specificity of 13% (95% Crl: 3.8-30.9).

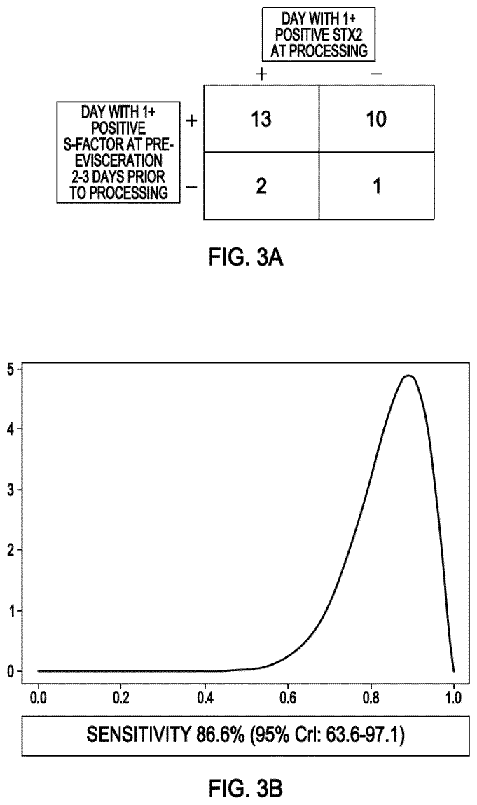

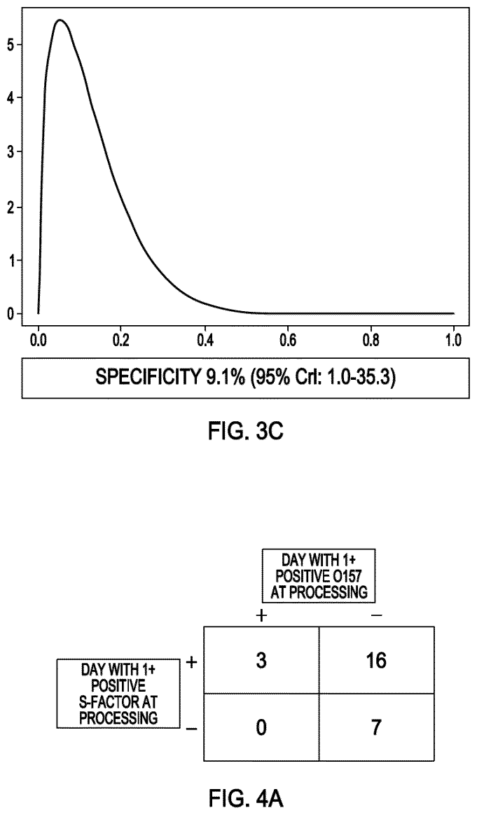

FIGS. 3A-C illustrate testing of S-factor at slaughter vs. testing of STX2 at processing, with FIG. 3A illustrating a testing grid showing days with one or more positive S-samples at pre-evisceration 2-3 days prior to process for STX2 positive or negative samples, FIG. 3B illustrating a sensitivity of 86.6% (95% Crl: 63.6-97.1), and FIG. 3C illustrating a specificity of 9.1% (95% Crl: 1.0-35.3).

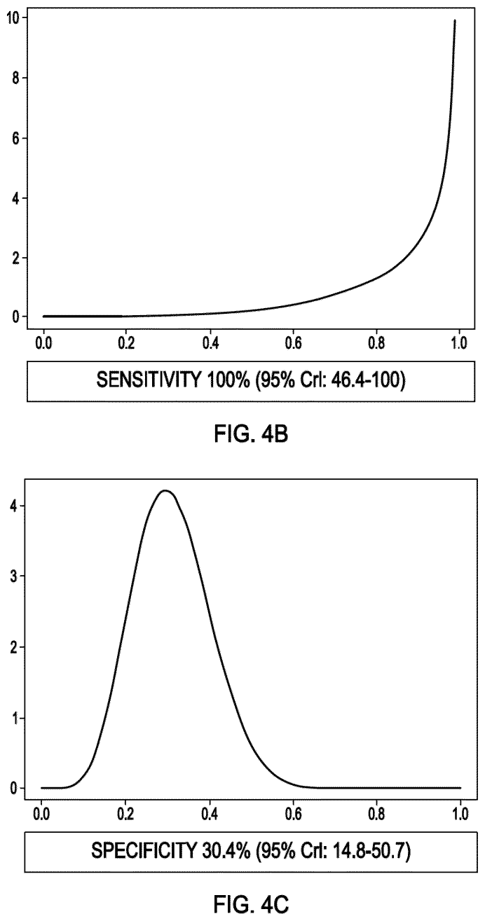

FIGS. 4A-C illustrate testing of S-factor vs. testing of O157 at processing, with FIG. 4A illustrating a testing grid showing days with one or more positive S-samples at processing for O157 positive or negative samples, FIG. 4B illustrating a sensitivity of 100% (95% Crl: 46.4-100), and FIG. 4C illustrating a specificity of 30.4% (95% Crl: 14.8-50.7).

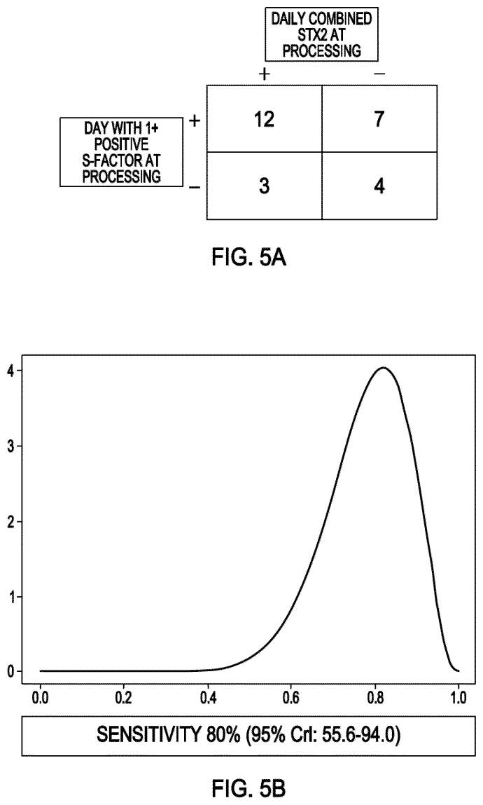

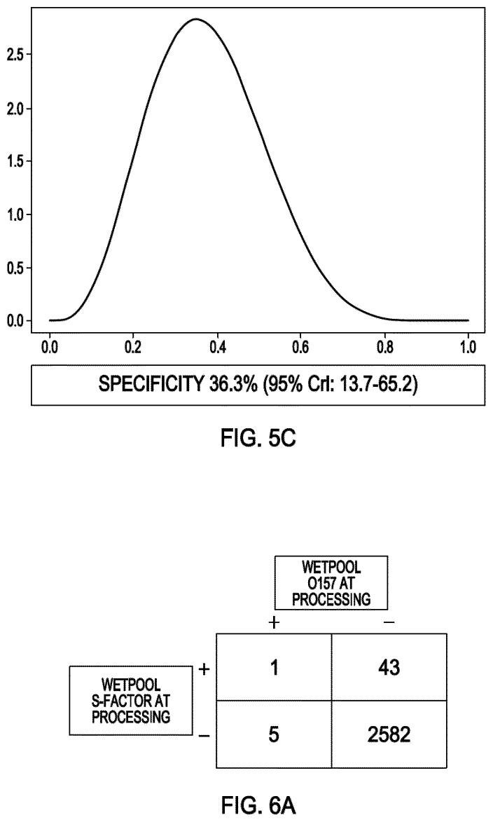

FIGS. 5A-C illustrate testing of S-factor vs. testing of STX2, with FIG. 5A illustrating a testing grid showing days with one or more positive S-samples at processing for STX2 positive or negative samples, FIG. 5B illustrating a sensitivity of 80% (95% Crl: 55.6-94.0), and FIG. 5C illustrating a specificity of 36.3% (95% Crl: 13.7-65.2).

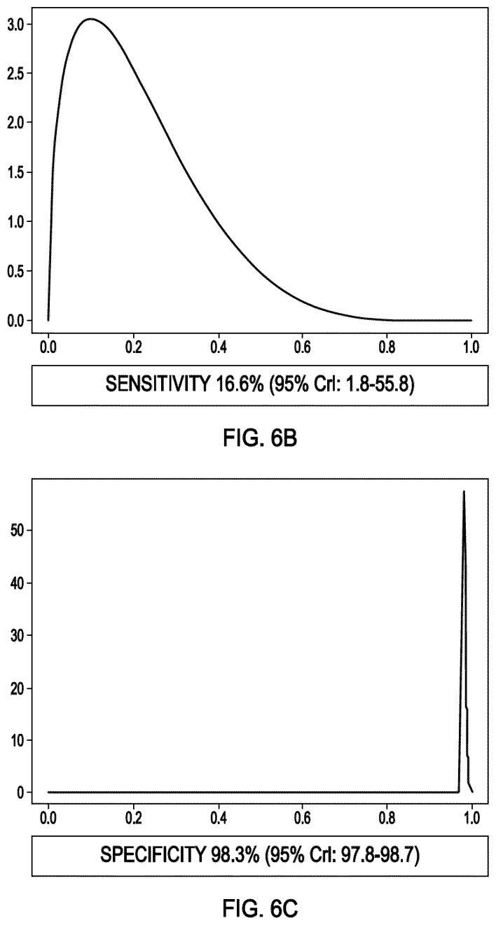

FIGS. 6A-C illustrate testing of S-factor vs. testing of O157 at processing using wet pool samples, with FIG. 6A illustrating a testing grid showing days with one or more positive wet pool S-samples at processing for wet pool O157 positive or negative samples, FIG. 6B illustrating a sensitivity of 16.6% (95% Crl: 1.8-55.8), and FIG. 6C illustrating a specificity of 95.3% (95% Crl: 97.8-98.7).

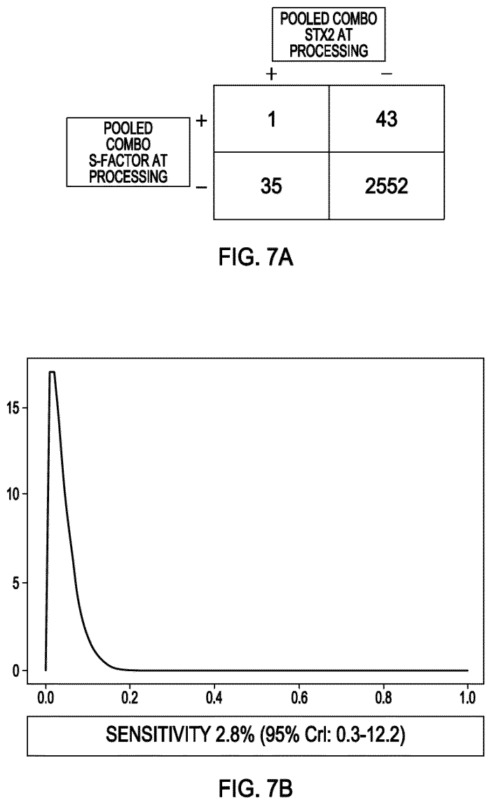

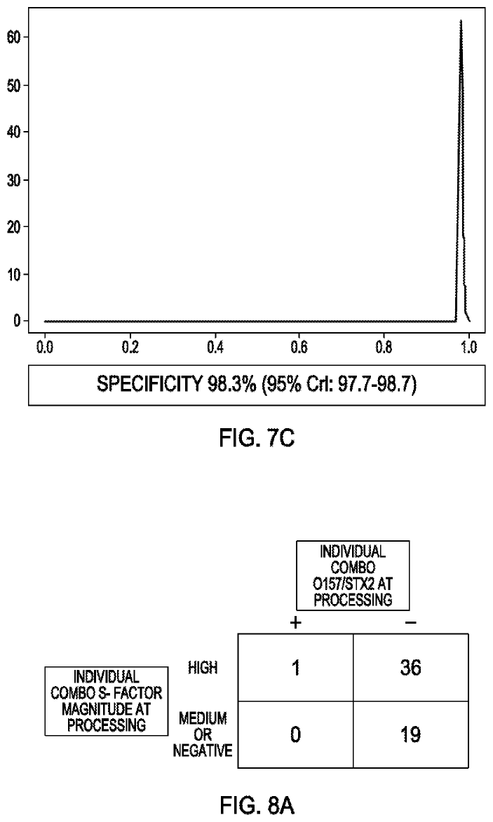

FIGS. 7A-C illustrate testing of S-factor vs. testing of STX2 at processing using wet pool samples, with FIG. 7A illustrating a testing grid showing days with one or more positive wet pool S-samples at processing for wet pool STX2 positive or negative samples, FIG. 7B illustrating a sensitivity of 2.8% (95% Crl: 0.3-12.2), and FIG. 7C illustrating a specificity of 98.3% (95% Crl: 97.7-98.7).

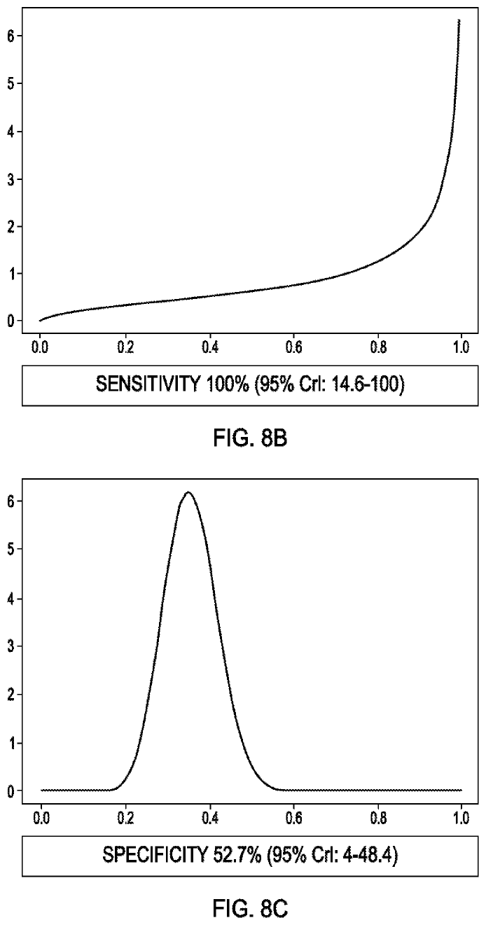

FIGS. 8A-C illustrate testing of S-factor vs. testing of O157/STX2 at processing, as follow-up testing on individuals from positive wet pool, with FIG. 8A illustrating a testing grid showing days with high or medium/negative magnitude individual S-factor for O157/STX2 positive or negative samples, FIG. 8B illustrating a sensitivity of 100% (95% Crl: 14.6-100), and FIG. 8C illustrating a specificity of 52.7% (95% Crl: 23.4-48.4).

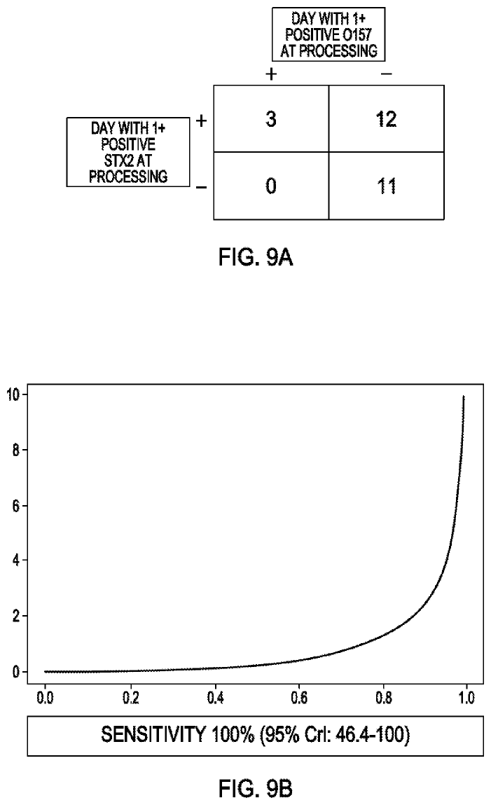

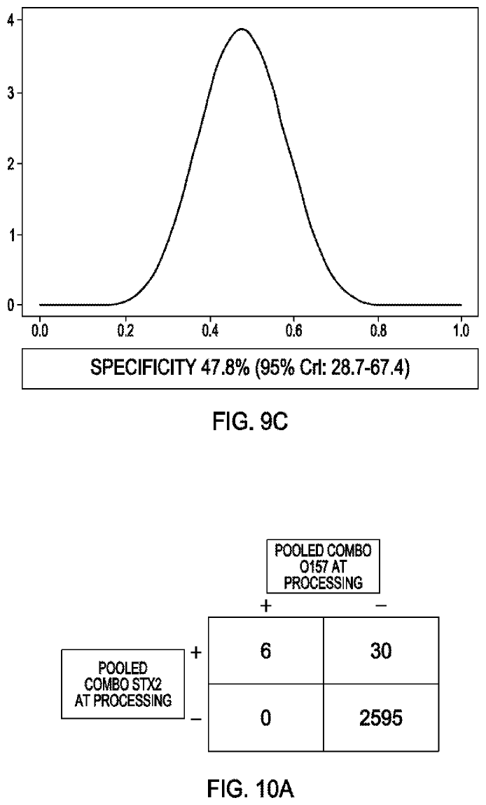

FIGS. 9A-C illustrate testing of STX2 versus O157, with FIG. 9A illustrating a testing grid showing days with at least one positive STX2 at processing for O157 positive or negative samples, FIG. 9B illustrating a sensitivity of 100% (95% Crl: 46.4-100), and FIG. 9C illustrating a specificity of 47.8% (95% Crl: 28.7-67.4).

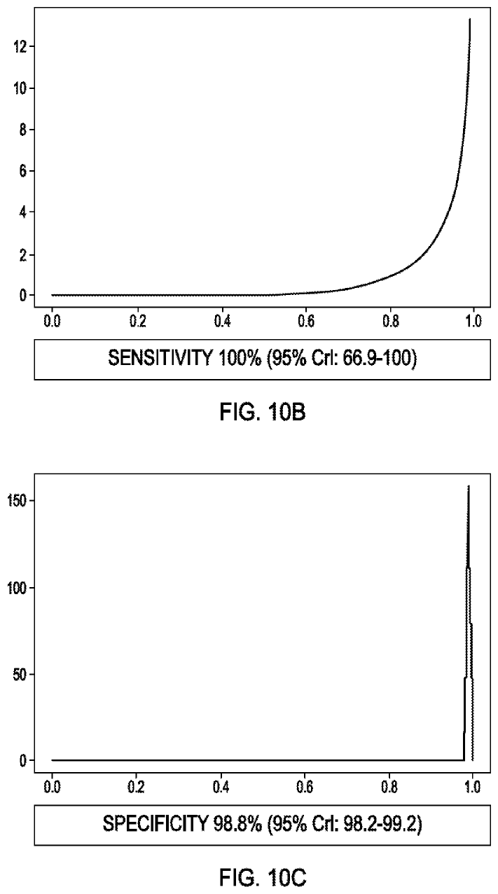

FIGS. 10A-C illustrate testing of STX2 versus O157 using wet pool samples, with FIG. 10A illustrating a testing grid showing days with one or more positive wet pool S-samples at processing for O157 positive and negative samples, FIG. 10B illustrating a sensitivity of 100% (95% Crl: 66.9-100), and FIG. 10C illustrating a specificity of 98.8% (95% Crl: 98.2-99.2).

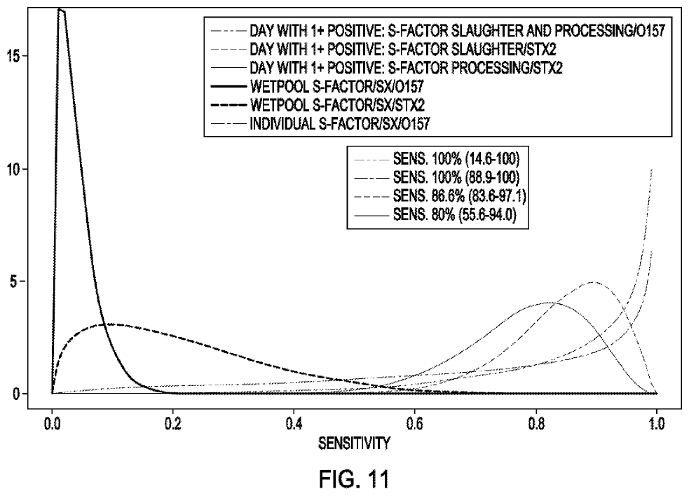

FIG. 11 illustrates days with one or more positive test using various testing protocols versus sensitivity.

Days with one or more positive S-sample tests at pre-evisceration has the best sensitivity to predict days with 1—STX2 positive test in trim 2-3 days later, relative to other testing combinations explored in this Example. Comparing STX2 to O157, STX2 had a higher false positive rate of 47.8% (95% Crl: 28.7-67.4).

Days with one or more positive S-sample tests has better sensitivity to predict processing days with one or more STX2 or O157 positive test.

Wet pool samples generally had better specificity to predict negative STX2/O157 wet pool samples. A high number of wet pool samples results in low specificity and high uncertainty. STX2 had high sensitivity in predicting O157 results, but high uncertainty.

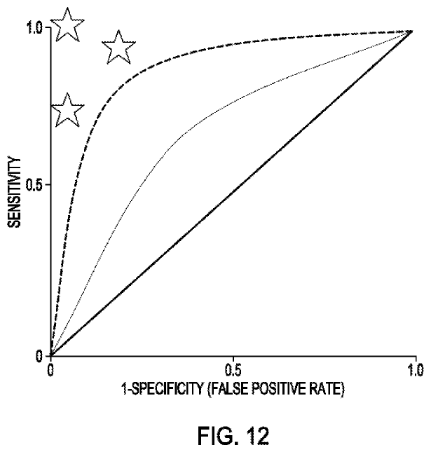

FIG. 12 illustrates sensitivity versus 1-specificity (e.g., false positive rate). With respect to threshold cycle (Ct) values and receiver operating curve (ROC), there is a potential for using quantitative Ct values to determine threshold, such as to balance sensitivity and specificity by identifying an optimized or improved cut-off value. The Ct values used to determine threshold can be adjusted to achieve various screening goals, and can justify different sampling protocols such as varying the frequency of sampling, the location of sampling along the production chain, and/or the use of single tests vs. testing in series or parallel.

### Example 2. Operation Design. Possible Protocol S-Target Carcass Testing to Predict E. Coli O157:H7 in Final Product of a Beef Processing Facility

### Materials and Methods

Samples will be collected over a production day at a commercial feed-beef processing plant. Ten to sixty samples per day will be collected from pre-evisceration sample sites during production day. Samples will be collected as described in the carcass mapping procedure at the location point 2: pre-evisceration. Cattle lots will be sampled for carcass S-target.

### Sample Collection

All samples will be obtained using SpeciSponges (Nasco, Fort Atkinson, Wis.) moistened with 10 ml of buffered peptone water (Difco Laboratories, Sparks, Md.). Excess liquid will be squeezed from the sponge into the sponge bag prior to sampling and then used to swab the carcass. The carcass sample will be collected from a 4000-cm² area. flipping the sponge over midway through taking the sample. One sample includes approximately five vertical and five horizontal passes (up and down or side to side will be considered one pass). For each of the two carcass processing points, two separate 4,000-cm² areas will be sampled and analyzed separately. Sampling at the pre-evisceration will be facilitated by using two sponges, each moistened with 10 mL of buffered peptone water. One sponge will be used for the inside and outside round area (carcass high) and one for the navel-plate-brisket-foreshank area (carcass low). All sample bags will be held refrigerated (2-10° C.) prior to analysis. Sampled carcass will not be held.

### Sample Analysis

The testing laboratory will add an additional 10 mL of Buffered Peptone Water to each sponge sample prior to plating. Each sponge will be analyzed S-target. S-target analysis will be performed using the Hygiena BAX S-target assay.

### Operation Actions

At a S-target enumeration level or prevalence of S-target at the carcass level action will be taken to adjust operation (line speed, employee audits, intervention applications or possible reprocessing carcass).

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects

The following exemplary aspects are provided, the numbering of which is not to be construed as designating levels of importance:

Aspect 1 provides a method of predicting microbial contamination, the method comprising:
gathering a sample from one or more carcasses;
performing a test for the microbial contamination on the sample to generate test results comprising detection of one or more microbial genetic markers of the microbial contamination;
predicting from the test results whether the one or more carcasses comprise the microbial contamination; and
releasing the one or more carcasses for further processing.

Aspect 2 provides the method of Aspect 1, wherein releasing the one or more carcasses for further processing comprises performing bacterial reduction processing on the one or more carcasses if the one or more carcasses are predicted to comprise the microbial contamination, and optionally performing bacterial reduction processing on the one or more carcasses if the one or more carcasses are predicted to not comprise the microbial contamination. Bacterial reduction processing can include cooking, irradiation, high pressure processing, thermal carcass surface treatment, steam carcass surface treatment, hot water carcass surface treatment, carcass surface treatment with one or more antimicrobial compounds, or a combination thereof.

Aspect 3 provides the method of any one of Aspects 1-2, wherein releasing the one or more carcasses for further processing comprises performing further testing for the microbial contamination on the one or more carcasses or processing products thereof and releasing the one or more carcasses into a stream of commerce for consumption if the further testing indicates absence of the microbial contamination.

Aspect 4 provides the method of any one of Aspects 1-3, wherein releasing the one or more carcasses for further processing comprises releasing the one or more carcasses into a stream of commerce for consumption, fabricating the one or more carcasses into raw products for consumption, or a combination thereof.

Aspect 5 provides the method of any one of Aspects 1-4, wherein the method is free of holding carcasses based on the test results.

Aspect 6 provides the method of any one of Aspects 1-5, wherein the carcass is a beef carcass, a poultry carcass, a pork carcass, a fish carcass, or a lamb carcass.

Aspect 7 provides the method of any one of Aspects 1-6, wherein the carcass is a beef carcass.

Aspect 8 provides the method of any one of Aspects 1-7, wherein the one or more carcasses include a hide.

Aspect 9 provides the method of Aspect 8, wherein the hide is an open hide.

Aspect 10 provides the method of any one of Aspects 1-7, wherein the one or more carcasses are free of a hide.

Aspect 11 provides the method of any one of Aspects 1-10, wherein the one or more carcasses are pre-eviscerated.

Aspect 12 provides the method of any one of Aspects 1-10, wherein the one or more carcasses are eviscerated.

Aspect 13 provides the method of any one of Aspects 1-12, wherein the one or more carcasses are trimmed.

Aspect 14 provides the method of any one of Aspects 1-13, wherein the one or more carcasses are free of trim pieces.

Aspect 15 provides the method of any one of Aspects 1-14, wherein the microbial contamination comprises bacterial, fungal, viral, and/or parasitic pathogens; indicator organisms (e.g., heterotrophes, generic E. coli, and/or total and fecal coliforms and enterococcus); spoilage organisms (e.g., Pseudomonas); indicator molecules (e.g., glial fibillary acid protein (GFAP)); transmissible spongiform encephalopathy (TSE) agents (prions) (e.g., bovine spongiform encephalopathy (BSE) agents, scrapie, and/or chronic wasting disease); and/or combinations thereof.

Aspect 16 provides the method of any one of Aspects 1-15, wherein the microbial contamination comprises Escherichia coli O157:H7 (E. coli O157:H7), enterohemorrhagic Escherichia coli (EHEC), enterotoxigenic Escherichia coli (ETEC), enteroinvasive Escherichia coli (EIEC), enterpathogenic Escherichia coli (EPEC), Salmonella, Listeria, Yersinis, Campylobacter, Staphylococcus spp., and/or Clostridial species.

Aspect 17 provides the method of any one of Aspects 1-16, wherein the microbial contamination comprises Escherichia coli O157:H7 (E. coli O157:H7).

Aspect 18 provides the method of any one of Aspects 1-17, wherein a production lot comprises the one or more carcasses.

Aspect 19 provides the method of Aspect 18, wherein the one or more carcasses are first one or more carcasses, further comprising performing the method on a second one or more carcasses in the production lot.

Aspect 20 provides the method of Aspect 19, comprising performing the method on all the carcasses in the production lot.

Aspect 21 provides the method of Aspect 19, comprising performing the method on less than all the carcasses in the production lot.

Aspect 22 provides the method of any one of Aspects 19-21, wherein gathering the sample from the second one or more carcasses in the production lot comprises combining the sample from the second one or more carcasses in the production lot with the sample from the first one or more carcasses in the production lot.

Aspect 23 provides the method of any one of Aspects 19-22, wherein gathering the sample from the second one or more carcasses in the production lot comprises swabbing an exterior of the second one or more carcasses in the production lot used to swab an exterior of the first one or more carcasses in the production lot.

Aspect 24 provides the method of any one of Aspects 19-21, wherein the method comprises maintaining and testing the sample from the first one or more carcasses and a sample from the second one or more carcasses.

Aspect 25 provides the method of any one of Aspects 19-21, wherein the method is free of combining the sample from the first one or more carcasses and a sample from the second one or more carcasses.

Aspect 26 provides the method of any one of Aspects 1-25, wherein gathering the sample comprises gathering the sample from an exterior of the one or more carcasses. Gathering the sample can be free of gathering the sample from an interior of the one or more carcasses. The method can be free of gathering the sample, or any sample, from an interior of any of the one or more carcasses. The gathering the sample can consist of gathering the sample from an exterior of the one or more carcasses.

Aspect 27 provides the method of Aspect 26, wherein gathering the sample comprises gathering the sample from 50 cm² to 10,000 cm² of a surface area of the exterior of the one or more carcasses.

Aspect 28 provides the method of any one of Aspects 26-27, wherein gathering the sample comprises gathering the sample from 1% to 95% of a surface area of the exterior of the one or more carcasses.

Aspect 29 provides the method of any one of Aspects 26-28, wherein gathering the sample comprises gathering the sample from a hide on the one or more carcasses, a de-hided portion of the one or more carcasses, an eviscerated portion of the one or more carcasses, a trimmed portion of the one or more carcasses, or a combination thereof.

Aspect 30 provides the method of any one of Aspects 1-29, wherein gathering the sample comprises swabbing an exterior of the one or more carcasses.

Aspect 31 provides the method of any one of Aspects 1-30, wherein gathering the sample comprises swabbing an exterior of the one or more carcasses with one or more sponges.

Aspect 32 provides the method of any one of Aspects 1-31, wherein the sample comprises a sample from one carcass and not more than one carcass.

Aspect 33 provides the method of any one of Aspects 1-31, wherein the sample comprises a sample from more than one carcass.

Aspect 34 provides the method of Aspect 33, wherein the sample comprises a sample from more than one carcass gathered in series.

Aspect 35 provides the method of any one of Aspects 33-34, wherein the sample comprises a sample from more than one carcass gathered at least partially in parallel.

Aspect 36 provides the method of any one of Aspects 1-35, wherein the test for the microbial contamination comprises a test that detects one and not more than one microbial genetic marker of the microbial contamination.

Aspect 37 provides the method of any one of Aspects 1-36, wherein the test for the microbial contamination comprises a test that detects one or more microbial genetic markers of Escherichia coli O157:H7 (E. coli O157:H7).

Aspect 38 provides the method of any one of Aspects 1-37, wherein the test for the microbial contamination comprises a test that detects one and not more than one microbial genetic marker of Escherichia coli O157:H7 (E. coli O157:H7).

Aspect 39 provides the method of any one of Aspects 1-38, wherein the one or more microbial genetic markers of the microbial contamination comprise one or more genetic markers from one or more microorganisms that are indicative of the microbial contamination.

Aspect 40 provides the method of Aspect 39, wherein the one or more microbial genetic markers of the microbial contamination comprise one or more genetic markers from one or more gram negative organisms in the Enterobacteriaceae family.

Aspect 41 provides the method of Aspect 40, wherein the one or more gram negative organisms in the Enterobacteriaceae family are other than the predicted microbial contamination.

Aspect 42 provides the method of any one of Aspects 1-41, wherein the one or more microbial genetic markers of the microbial contamination comprise one or more genetic markers from more than one microorganism that are indicative of the microbial contamination.

Aspect 43 provides the method of any one of Aspects 1-41, wherein the one or more microbial genetic markers of the microbial contamination comprise one or more genetic markers from a single microorganism that is indicative of the microbial contamination.

Aspect 44 provides the method of any one of Aspects 1-43, wherein the one or more microbial genetic markers of the microbial contamination comprise a genetic marker from one or more microorganisms that are indicative of microbial contamination with Escherichia coli O157:H7 (E. coli O157:H7).

Aspect 45 provides the method of Aspect 44, wherein the one or more microorganisms that are indicative of microbial contamination with Escherichia coli O157:H7 comprise one or more gram negative organisms in the Enterobacteriaceae family other than Escherichia coli O157:H7.

Aspect 46 provides the method of any one of Aspects 1-45, wherein the test for the microbial contamination comprises an S-target test, a PCR assay for O157, a PCR assay for STX2, or a combination thereof.

Aspect 47 provides the method of any one of Aspects 1-46, wherein the test for the microbial contamination comprises an S-target test.

Aspect 48 provides the method of any one of Aspects 1-47, wherein predicting whether the one or more carcasses comprise the microbial contamination comprises predicting that the one or more carcasses comprise the microbial contamination if the results of the test indicate presence of the one or more microbial genetic markers of the microbial contamination in the sample, and predicting that the one or more carcasses do not comprise the microbial contamination if the results of the test indicate no detectable presence of the one or more microbial genetic markers of the microbial contamination.

Aspect 49 provides the method of any one of Aspects 1-48, wherein predicting whether the one or more carcasses comprise the microbial contamination comprises using a correlation between the test for the microbial contamination and a positive test for the microbial contamination from a different test for the microbial contamination to predict whether the test results indicate that the one or more carcasses comprise the microbial contamination.

Aspect 50 provides the method of Aspect 49, further comprising generating the correlation between the test for the microbial contamination and the positive test for the microbial contamination from the different test from the microbial contamination.

Aspect 51 provides the method of any one of Aspects 49-50, wherein the correlation comprises a percent certainty based on a degree of positivity of the detection of the one or more microbial genetic markers, wherein predicting whether the one or more carcasses comprises the microbial contamination comprises predicting that the test results indicate that the one or more carcasses comprise the microbial contamination if the percent certainty of the correlation indicated by the positivity of the detection of the one or more microbial genetic markers equals or exceeds a pre-determined threshold percent certainty, and predicting that the test results indicate that the one or more carcasses do not comprise the microbial contamination if the percent certainty of the correlation indicated by the positivity of the detection of the one or more microbial genetic markers is less than the pre-determined threshold percent certainty.

Aspect 52 provides a method of predicting microbial contamination, the method comprising:
gathering a sample from one or more carcasses, comprising swabbing an exterior of the one or more carcasses;
performing a test for the microbial contamination on the sample to generate test results comprising detection of one or more microbial genetic markers of the microbial contamination, wherein the one or more microbial genetic markers of the microbial contamination comprises one or more genetic markers from one or more gram negative organisms in the Enterobacteriaceae family other than the predicted microbial contamination;
predicting from the test results whether the one or more carcasses comprise the microbial contamination; and
releasing the one or more carcasses for further processing.

Aspect 53 provides a method of predicting microbial contamination from Escherichia coli O157:H7 (E. coli O157:H7), the method comprising:
gathering a sample from one or more carcasses, comprising swabbing an exterior of the one or more carcasses;
performing a test for the microbial contamination on the sample to generate test results comprising detection of one or more microbial genetic markers of the microbial contamination, wherein the one or more microbial genetic markers of the microbial contamination comprise one or more genetic markers from one or more gram negative organisms in the Enterobacteriaceae family other than E. coli O157:H7;
predicting from the test results whether the one or more carcasses comprise the microbial contamination; and
releasing the one or more carcasses for further processing.

Aspect 54 provides the method of any one or any combination of Aspects 1-53 optionally configured such that all elements or options recited are available to use or select from.

What is claimed is:

1. A method of processing carcasses that may be contaminated by Escherichia coli O157:H7 (E. coli O157:H7), the method comprising:
gathering a sample from one or more carcasses;
performing a test for microbial contamination on the sample to generate test results comprising detection of one or more microbial genetic markers of one or more gram negative organisms in the Enterobacteriaceae family other than E. coli O157:H7;
predicting from the test results whether the one or more carcasses is contaminated by E. coli O157:H7; and
releasing the one or more carcasses for further processing.

2. The method of claim 1, wherein releasing the one or more carcasses for further processing comprises performing bacterial reduction processing on the one or more carcasses if the one or more carcasses are predicted to be contaminated by E. coli O157:H7.

3. The method of claim 2, wherein performing bacterial reduction processing comprises cooking, irradiation, high pressure processing, thermal carcass surface treatment, steam carcass surface treatment, hot water carcass surface treatment, carcass surface treatment with one or more antimicrobial compounds, or a combination thereof.

4. The method of claim 1, wherein releasing the one or more carcasses for further processing comprises performing further testing on the one or more carcasses predicted to be contaminated by E. coli O157:H7, or processing products thereof, for contamination by E. coli O157:H7 and releasing the carcasses predicted to be contaminated by E. coli O157:H7, or the processing products, into a stream of commerce for consumption if the further testing indicates absence of contamination from E. coli O157:H7.

5. The method of claim 1, wherein the method is free of holding carcasses based on the test results.

6. The method of claim 1, wherein the carcass is a beef carcass.

7. The method of claim 1, wherein the one or more carcasses include a hide, the one or more carcasses are pre-eviscerated, or the one or more carcasses are post-eviscerated.

8. The method of claim 1, wherein performing the test for the microbial contamination further comprises detection of bacterial, fungal, viral, and/or parasitic pathogens other than said gram negative organisms in the Enterobacteriaceae family; indicator organisms; spoilage organisms; indicator molecules; transmissible spongiform encephalopathy (TSE) agents (prions); or a combination thereof.

9. (canceled)

10. The method of claim 1, wherein a production lot comprises the one or more carcasses, wherein the one or more carcasses are first one or more carcasses, further comprising performing the method on a second one or more carcasses in the production lot.

11. (canceled)

12. The method of claim 1, wherein gathering the sample comprises gathering the sample from an exterior of the one or more carcasses.

13. The method of claim 1, wherein the method is free of gathering any sample from an interior of any of the one or more carcasses.

14. The method of claim 1, wherein gathering the sample comprises gathering the sample from a hide on the one or more carcasses, a de-hided portion of the one or more carcasses, an eviscerated portion of the one or more carcasses, a trimmed portion of the one or more carcasses, or a combination thereof.

15. The method of claim 1, wherein gathering the sample comprises swabbing an exterior of the one or more carcasses.

16-18. (canceled)

19. The method of claim 1, wherein the test for the microbial contamination comprises a PCR assay that detects the one or more microbial genetic markers.

20. The method of claim 1, wherein predicting whether the one or more carcasses is contaminated by E. coli O157:H7 comprises using a correlation between the test for the microbial contamination and a positive test for different microbial contamination from a different test for different microbial contamination to predict whether the test results indicate that the one or more carcasses is contaminated by E. coli O157:H7.

21. A method of predicting microbial contamination, the method comprising:
gathering a sample from one or more carcasses, comprising swabbing an exterior of the one or more carcasses;
performing a test for the microbial contamination on the sample to generate test results comprising detection of one or more microbial genetic markers of the microbial contamination, wherein the one or more microbial genetic markers of the microbial contamination comprises one or more genetic markers from one or more gram negative organisms in the Enterobacteriaceae family other than the predicted microbial contamination;
predicting from the test results whether the one or more carcasses comprise the microbial contamination; and
releasing the one or more carcasses for further processing.

22. A method of predicting microbial contamination from Escherichia coli O157:H7 (E. coli O157:H7), the method comprising:
gathering a sample from each of at least two carcasses, comprising swabbing an exterior of each of the carcasses;
performing a test for the microbial contamination from E. coli O157:H7 on each sample to generate test results comprising detection of one or more microbial genetic markers from one or more gram negative organisms in the Enterobacteriaceae family other than E. coli O157:H7; and
predicting from the test results whether the carcass from which the sample was taken is contaminated by E. coli O157:H7.

23. The method of claim 1, wherein a sample is gathered from each of at least two carcasses, the test is performed on each of the samples, the prediction is done for each carcass from the sample from which it was taken, and each of the carcasses is released for further processing.

24. The method of claim 23 wherein releasing the carcasses for further processing comprises performing bacterial reduction processing on each carcass predicted to be contaminated by E. coli O157:H7.

25. The method of claim 23 wherein releasing the carcasses for further processing comprises performing further testing on the one or more carcasses predicted to be contaminated by E. coli O157:H7, or processing products thereof, for contamination by E. coli O157:H7 and releasing the carcasses predicted to be contaminated by E. coli O157:H7, or the processing products, into a stream of commerce for consumption if the further testing indicates absence of contamination from E. coli O157:H7.
